(19) United States
(12) Patent Application Publication
(10) Pub. No.: US 2014/0213832 A1
(43) Pub. Date: Jul. 31, 2014

(54) Stable Spherical, Porous Metal-Organic Framework Shaped Bodies For Gas Storage And Gas Separation

(71) Applicant: BASF SE, Ludwigshafen (DE)

(72) Inventors: Manuela Gaab, Heidelberg (DE); Milan Kostur, Mutterstadt (DE); Ulrich Müller, Neustadt (DE)

(73) Assignee: BASF SE, Ludwigshafen (DE)

(21) Appl. No.: 14/169,895

(22) Filed: Jan. 31, 2014

(30) Foreign Application Priority Data
Jan. 31, 2013 (EP) 13153514.8

(51) Int. Cl. B01J 20/30; C07C 7/12; B01J 20/28

(52) U.S. Cl. CPC B01J 20/3078; B01J 20/28019; C07C 7/12
USPC 585/3; 502/8; 502/62; 585/830

(57) ABSTRACT

The present invention relates to a method for preparing a MOF shaped body in the form of spheres, MOF shaped bodies in the form of spheres and a method of uptake of at least one substance for the purposes of its storage, separation, controlled release, chemical reaction or as support utilizing MOF shaped bodies in the form of spheres.

### CROSS-REFERENCE TO RELATED APPLICATIONS

This application claims the benefit of priority under 35 U.S.C. §119(a) to European Patent (EP) Application No. 13153514.8 filed Jan. 31, 2013, which is incorporated by reference in its entirety for all purposes

### FIELD OF THE INVENTION

Principles and embodiments of the present invention relate to pulverulent materials for compact shaped bodies.

### BACKGROUND OF THE INVENTION

Due to their large surface areas of up to 10 000 m²/g, metal-organic framework (MOF) materials are of interest for applications in gas storage or gas separation. For most applications, it is necessary to process the pulverulent materials to compact shaped bodies. These can be handled more conveniently and especially in a safer manner, allow better exploitation of the apparatus or tank volumes and prevent large pressure drops. Prerequisites for the successful use of such shaped bodies are, however, the absorption capacity and selectivity thereof, adequate thermal and mechanical stability and high abrasion resistance. Even the recurrent thermal shocks resulting from the heat of adsorption released in the course of continuous adsorption/desorption cycles can be sufficient in the case of the related zeolite shaped bodies to cause fracture and splintering of the bodies (DE 1 905 019). Mechanical stability is therefore indispensible particularly for MOF shaped bodies which are used in vehicle tanks exposed constantly to agitation.

The chemical and/or physical mechanisms in the course of compaction and setting of metal-organic framework materials are not nearly as well understood as is the case for the related class of the zeolites, and so the results which are achieved with new additives and shaped bodies can be predicted only with low certainty.

Spheres have particularly high stability since curve shapes distribute pressure exerted and thus withstand relatively high forces (cf. egg). As a result of the lack of edges as occur, for example, in the case of extrudates or tablets, the risk that material parts will splinter off under mechanical stress on the spheres is minimized.

The production of MOF tablets and extrudates has been described in WO 2003/102000 and WO 2006/050898.

The use of MOF spheres of Cu-BTC (diameter 2-3 mm) has been described in a publication by M.G. Plaza et al. in Separation and Purification Technology 90 (2012) 109-119 for the separation of propane and propene. The publication refers to the production of the Cu-BTC powder. However, there is no explanation as to the manner in which the Cu-BTC spheres were produced. Nor is there any reference to other sources in which the sphere preparation is described.

Chem. Commun. 48 (2012) 9388-9390 discloses core-shell spheres which are formed by using ca. 3 μm mesoporous silica spheres as the core onto which a shell of zeolite imidazolate frameworks, so-called ZIF-8 is grown.

In Nature Chemistry 3 (2011) 347-348 the formation of hollow spheres is described.

WO2012/156436 describes the formation of MOF spheres by a gelation process from a MOF-gel precursor solution. The use of a binder is minimized in order to avoid blocking of the pores and the respective effects, e.g. decreasing specific surface and pore volume. The resulting MOF particles are obtained in the form of a dried gel (xerogel or aerogel).

Typical processes in the state of the art for producing shaped bodies include extrusion, tableting, kneading, pan milling and shaping. Kneading and/or pan milling and shaping can be carried out by any suitable method, for example as described in

Ullmanns Enzyklopädie der Technischen Chemie, 4th edition, Volume 2, p. 313 ff. (1972).

### BRIEF SUMMARY OF THE INVENTION

Principles and embodiments of the present invention relate to mechanically stable, spheroidal MOF shaped bodies with high surface areas and high adsorption capacities. These may be used for example in gas storage and/or gas separation and can be produced via an industrially implementable, favorable production process. Some applications may include the storage and/or separation of natural gas or shale gas, for example the storage of natural gas or shale gas in vehicle tanks.

Embodiments of the invention relate to a method for preparing a shaped body in the form of spheres comprising the step of mixing a composition comprising the MOF and at least one liquid, wherein the liquid may be water. The method may further comprise mixing at least one additive with the composition, wherein the at least one additive comprises a binder, which can be selected from the group consisting of inorganic oxides, clays, and concrete, and wherein the amount of the at least one binder additive based on the total weight of the shaped body can be in a range from 1 to 80 wt.-%, or from 2 to 50 wt.-%, or from 3 to 30 wt.-%, or from 4 to 20 wt.-%, or from 5 to 10 wt.-%. The at least one additive may comprise a pore forming agent selected from the group consisting of organic polymers, wherein the organic polymer is selected from the group consisting of methylcellulose, polyethylene oxide, or mixtures thereof.

In various embodiments of the invention, a metal of the MOF is selected from the group consisting of Mg, Zn, and Al, or mixtures thereof. In one or more embodiments the metal of the MOF may be Al. The MOF may comprise aluminum; and fumarate, trimesate, 2-aminoterephthalic acid or 4,4′,4″-benzene-1,3,5-triyl-tribenzoate, or mixtures thereof.

Embodiments of the method may further comprise heating the composition at a temperature of 100° C. or less, or at a temperature of 80° C. or less, or at a temperature of 50° C. or less, or at a temperature between from 20° C. to 50° C.

Embodiments of the method may further comprise an activation step at a temperature of 300° C. or less, or at a temperature of 250° C. or less, or at a temperature of 200° C. or less.

Embodiments of the present invention also relate to a shaped body in the form of spheres produced by the method described herein. The spheres can have diameters in the range of from 1 mm to 50 mm, or from 1.5 mm to 30 mm, or from 2 mm to 20 mm, or from 2 mm to 15 mm.

Embodiments of the present invention also relate to a shaped body prepared in the form of spheres by various combinations of the method steps described herein.

Embodiments of the present invention also relate to a method for the uptake of at least one substance for the purposes of its storage, separation, controlled release, chemical reaction or as support, comprising providing a shaped body as described herein, and contacting the shaped body with the at least one substance, wherein the shaped body may be spherical.

Embodiments of the method may further comprise preparing the shaped body by mixing a composition comprising a MOF and at least one liquid and heating the mixture at a temperature of 100° C. or less by a method of any one of claims 1 to 10 for the uptake of at least one substance for the purposes of its storage, separation, controlled release, chemical reaction or as support, wherein the at least one substance is a gas or gas mixture. The at least one substance can be natural gas or shale gas

The shaped body can be introduced into a vehicle tank, a gas container, or a storage volume of a gas transporter vehicle, and is brought into contact with the at least one substance for storage in the vehicle tank, gas container, or storage volume of the gas transporter vehicle.

### DETAILED DESCRIPTION OF THE INVENTION

Established industrial processes for the production of spherical bodies of porous materials (namely zeolites and molecular sieves) are based on the use of intensive mixers, granulating pans, ballformers or marumerizers and binder additives. It has been found that, surprisingly, typical binders as used for the inorganic zeolites (e.g. kaolin, attapulgite, bentonite, palygorskite, montmorillonite, sepiolite, silicates and mixtures thereof, described, inter alia, in U.S. Pat. No. 2,973,327, EP 0 940 174 and EP 1 468 731) also interact well with the semiorganic MOFs and lead to stable shaped bodies of appropriate hardness. Experts in the field expect that a switch to at least partly organic binders will be necessary, these having similar polarities to MOF frameworks and similar structures to the organic linkers thereof.

The hardness of the shaped bodies obtained in accordance with various embodiments of the invention is particularly surprising, since semiorganic MOFs, after the shaping step, cannot be calcined at the high temperatures typically required for zeolites (generally 500 to 600° C., e.g. EP 1 468 731). The high temperatures are required to form a ceramic from the binder used, this bringing about the hardness of the zeolite shaped body (typical crush strength around 40-50 N, e.g. EP 1 467 811). MOFs decompose at these high temperatures due to the proportion of organic units present. Surprisingly, even much lower temperatures (e.g. 200° C.) are sufficient to obtain shaped bodies of appropriate hardness.

Experts in the field avoid using binders to avoid pore blocking as is outlined in WO2012/156436. Additionally surprisingly, the conventional binders used according to embodiments of the present invention do not cause excessive conglutination or blockage of the highly porous MOF structures having up to 20 times the surface area of zeolites. The resulting spherical MOF shaped bodies have high surface areas and consequently exhibit high methane adsorptions. Particularly surprisingly, it is possible to add relatively high amounts of binder (e.g. 20% by weight) without any dramatic reduction in surface area. Normally, the adsorption capacity of the related zeolites is reduced by adding the above-described conventional binders (EP 1 467 811). In the case of MOF materials, the use of commercial cement as a binder actually leads to MOF spheres having application properties similar to those of the MOF powder. Embodiments of the inventive adsorption system thus, completely surprisingly, involve a wide range of standard (as in the case of zeolites) and unusual (e.g. cements) binder materials, and, for very different amounts of binder, very good application properties which can be adjusted precisely to the respective application via the type of binder used.

The inventive shaped bodies can be obtained by the process described with all kinds of MOF powders as described in the prior art and producible by the expert in the field. The inventive shaped bodies can have a somewhat oval to ideally spherical shape, in the form of smooth spheres or beads or with rough uneven surfaces. The spheroidal shaped bodies obtained in accordance with the principles and embodiments of the invention also have a relatively wide particle size distribution. By sieving, it is possible to separate the spheres into fractions with narrow particle size distribution, as is also common practice in the industrial production of established adsorbents (zeolites, molecular sieves).

Another embodiment of the present invention relates to a method for preparing a shaped body in the form of spheres comprising the step of mixing a composition comprising the MOF and at least one liquid.

As liquids, it is possible to use, inter alia, water or at least one alcohol such as, for example: a monoalcohol having from 1 to 4 carbon atoms, for example methanol, ethanol, n-propanol, isopropanol, 1-butanol, 2-butanol, 2-methyl-1-propanol or 2-methyl-2-propanol, or a mixture of water and at least one of the alcohols mentioned or a polyhydric alcohol such as a glycol, preferably a water-miscible polyhydric alcohol, either alone or as a mixture with water and/or at least one of the monohydric alcohols mentioned.

The at least one liquid may comprise water and/or aqueous solutions. In one or more embodiments the at least one liquid is water. In other embodiments the at least one liquid is a mixture of water and C₁ to C₄ organic alcohols.

In embodiments of the present invention, the ratio of MOF to the amount of liquid(s) (based on weight) may be for example in the range of from 1:0.1 to 1:10, the range also may be from 1:0.5 to 1:5, or in some embodiments from 1:1 to 1:4, or from 1:1.5 to 1:3.

It is important that the components are added in a certain order. First, at least part of the MOF is charged into the mixer and part of the at least one liquid is added. Later the remaining amounts of the MOF and the liquid are added sequentially to keep a certain humidity level in the mixture and let the granules consistently grow to spheres. In some embodiments of the present invention, the remaining amounts of the MOF and the liquid are dosed simultaneously.

The dosing rate is as such that the at least one liquid is always added in the form of a spray or droplets. The dosing rate may be in the range of from 0.1 liter per hour (l h⁻¹) to 100 l h⁻¹, or from 0.5 l h⁻¹ to 80 l h⁻¹, or from 1 l h⁻¹ to 30 h⁻¹, or from 1 l h⁻¹ to 10 l h⁻¹.

The term ‘mixing’ within the frame of this application is defined as follows: filling the components into a mixer and agitating the mixer.

Mixers comprise intensive mixers, rotary plates, marumerizers and any other equipment known to the expert. The mixers may be selected from the group consisting of intensive mixers, rotary plates, ballformers and marumerizers.

In another embodiment, the composition further comprises at least one additive, i.e. the concerning method comprises the step of mixing a composition comprising the MOF, the at least one liquid and at least one additive.

In an embodiment, the at least one additive comprises a binder, with the binder used basically being able to be any chemical compound which holds or draws other materials together to form a cohesive whole.

In one or more embodiments the at least one additive comprises a binder selected from the group consisting of inorganic oxides (for example, aluminum oxide), clays (for example, bentonite), and concrete.

Binders may be, for example, inter alia aluminum oxide or binders comprising aluminum oxide, as are described, for example, in WO 94/29408, silicon dioxide as described, for example, in EP 0 592 050 A1, mixtures of silicon dioxide and aluminum oxide as are described, for example, in WO 94/13584, clay minerals as are described, for example, in JP 03-037156 A, for example montmorillonite, kaolin, bentonite, halloysite, dickite, nacrite and anauxite, alkoxysilanes as are described, for example, in EP 0 102 544 B1, for example tetraalkoxysilanes such as tetramethoxysilane, tetraethoxysilane, tetrapropoxysilane, tetrabutoxysilane, or for example trialkoxysilanes such as trimethoxysilane, triethoxysilane, tripropoxysilane, tributoxysilane, alkoxytitanates, for example tetraalkoxytitanates such as tetramethoxytitanate, tetraethoxytitanate, tetrapropoxytitanate, tetrabutoxytitanate, or for example trialkoxytitanates such as trimethoxytitanate, triethoxytitanate, tripropoxytitanate, tributoxytitanate, alkoxyzirconates, for example tetraalkoxyzirconates such as tetramethoxyzirconate, tetraethoxyzirconate, tetrapropoxyzirconate, tetrabutoxyzirconate, or for example trialkoxyzirconates such as trimethoxyzirconate, triethoxyzirconate, tripropoxyzirconate, tributoxyzirconate, silica soles, cements and/or amphiphilic substances

Further additives which can be used during the mixing process and added at any time during the process are, inter alia, amines or amine derivatives such as tetraalkylammonium compounds or amino alcohols and carbonate-comprising compounds such as calcium carbonate. Such further additives are described, for instance, in EP 0 389 041 A1, EP 0 200 260 A1 or WO 95/19222. The order of the additives such as amines, binder, pasting agent, viscosity-increasing substance during mixing is in principle not critical.

Other additives comprise binders and/or pore forming agents. In an embodiment, the at least one additive comprises at least one binder. Binders may be selected from the group consisting of inorganic oxides (such as aluminum oxide), clays (such as bentonite), and concrete.

The amount of the at least one binder based on the total weight of the shaped body is from 1 to 80 wt.-%, or 2 to 50 wt.-%, or 3 to 30 wt.-%, or 4 to 20 wt.-%, or 5 to 10 wt.-%.

In a further embodiment the at least one additive comprises a pore forming agent. The pore forming agent may be selected from the group consisting of organic polymers, for example methylcellulose, polyethylene oxide or mixtures thereof.

The mixing can be carried out at elevated temperatures, for example in the range from room temperature to 300° C., and/or under superatmospheric pressure, for example in the range from atmospheric pressure to a few hundred bar, and/or in a protective gas atmosphere, for example in the presence of at least one noble gas, nitrogen or a mixture of two or more thereof.

An embodiment of a method according to the invention may be performed at a temperature of 100° C. or less, or at a temperature of 80° C. or less, or at a temperature of 50° C. or less, or between a temperature of from 20° C. to 50° C.

In a further embodiment, the shaped body obtained by mixing may be subjected to at least one drying step which is generally carried out at a temperature in the range from 25 to 500° C., or in the range from 50 to 500° C., or in the range from 100 to 350° C. It is likewise possible to carry out drying under reduced pressure or under a protective gas atmosphere.

In some embodiments, the shaped bodies may be heated after the mixing or the drying step in a so-called activation step. The activation step is performed at a temperature of 300° C. or less, or at a temperature of 250° C. or less, or at a temperature of 200° C. or less. Principles and embodiments of the present invention relate to MOFs wherein the metal of the MOF is selected from the group consisting of Mg, Zn, Al or mixtures thereof, and in a particular embodiment the metal is Al.

In another particular embodiment, the MOF comprises
aluminum; and
fumarate, trimesate, 2-aminoterephthalic acid or 4,4′,4″-benzene-1,3,5-triyltribenzoate or mixtures thereof.

The size of the shaped bodies that are yielded by the method are such that the smallest to largest diameters of the shaped bodies both are of from 1 to 50 mm, or for example from 1.5 to 30 mm, or from 2 to 20 mm, and may be from 2 to 15 mm. The minimum and maximum diameters can be determined using a sliding caliper.

By sieving, the spheres can be separated into fractions with narrow particle size distribution.

Principles and embodiments of the present invention also relate to a shaped body in the form of spheres obtainable by a method as described above.

Principles and embodiments also relate to the shaped bodies being suitable for storage of a gas.

A gas is a methane-containing mixture or methane. Another gas is hydrogen. A further gas is carbon dioxide (CO₂).

Principles and embodiments of the present invention also relate to a method for adsorbing, storing and/or releasing at least one gas by use of the metal-organic framework of embodiment of the invention as described herein.

Other principles and embodiments relate to the use of the shaped body for the uptake of at least one substance for the purposes of its storage, separation, controlled release, chemical reaction or as support. This concerns the use of the shaped body obtainable by a method as described before or obtained by a method as described before for the uptake of at least one substance for the purposes of its storage, separation, controlled release, chemical reaction or as support.

In some embodiments, the at least one substance is a gas or gas mixture, for example natural gas, shale gas or hydrogen.

In some particular embodiments, the at least one substance is natural gas or shale gas, which is stored in vehicle tanks or gas containers or gas transporters, such as ships and trucks.

Likewise, another embodiment of the present invention is accordingly a method of storing a gas, which comprises the step of bringing the gas into contact with a shaped body according to the embodiments of the invention.

Methane or methane-containing gases are particularly suitable for this storage.

Hydrogen is particularly suitable for this storage.

Carbon dioxide is also particularly suitable for this storage.

In addition, the shaped body of the embodiments of the invention is suitable for separating a gas from a gas mixture.

Further principles and embodiments of the present invention relate to a method for separating a gas from a gas mixture through the use of a shaped body according to the embodiments of the invention.

Likewise, a further embodiment of the present invention relates to a method of separating a gas from a gas mixture, which comprises the step of bringing a shaped body according to the invention into contact with the gas mixture.

For example, the gas mixture may, in particular, comprise methane and other gases, wherein the methane is preferably removed from the gas mixture.

Furthermore, the gas mixture may be a mixture comprising methane and water. Preference is given to removing gaseous water from the gas mixture. The gas mixture can be, for example, water-comprising natural gas. Other gases or volatile components which are preferably separated off are sulfur-based impurities in natural gas or shale gas like hydrogen sulfide or carbonyl sulfide.

Likewise, the gas mixture can be a gas mixture comprising hydrogen.

Likewise, the gas mixture can be a gas mixture comprising carbon dioxide.

The principles and embodiments of the present invention are illustrated by means of the examples below.

### EXAMPLES

The examples which follow describe the inventive spheronizing of MOF material. The MOF material used was produced according to WO 12/042410.

The spheroidal shaped bodies obtained had a relatively wide particle size distribution. For each example, the minimum and maximum diameters are reported as determined using a sliding caliper. By sieving, the spheres can be separated into fractions with narrow particle size distribution.

Bulk densities of sphere packings were determined using a jolting volumeter type STAV II from J. Engelsmann A G. The machine has been tested according to DIN ISO 787 by the manufacturer. A weighed amount of the respective sample was put into a 1000 or 100 mL scaled cylinder. After tapping the cylinder 3000 times, the resulting volume of the packing was determined and the density calculated by dividing sample weight by sample volume.

The density of spheres was determined by weighing a selected sphere, measuring its diameter with a sliding caliper and then dividing weight by volume (the latter being calculated via the diameter).

The specific surface area of the spheres was calculated by applying the Langmuir model according to DIN 66131 and 66134.

The crush strength is defined within the meaning of the various embodiments of the present invention as lateral pressure resistance to pressure and can be measured with a hardness grading device by Zwick.

### Example 1

### Spheronizing with 20% by weight of K10 clay

Aluminum fumarate MOF (1000 g) was initially charged in an Eirich intensive mixer (model: R02, RV02). K10 clay (250 g) was added and mixed with the MOF. A manual pressure sprayer was used to spray on demineralized water (2200 g) with continuous movement of the mixture over 50 minutes. Within this time, a second portion of aluminum fumarate MOF (160 g) was added. After completing the addition of water, the spherical shaped bodies formed were dried (12 h, 100° C.) and activated (5 h, 200° C.). 1058 g of spheres were obtained.

Diameter: 2-15 mm

Bulk density of sphere packing: 0.54 g/ml

Average density of spheres: 0.57-1.10 g/ml

Crush strength: 80 N

Langmuir surface area: 952 m2/g (surface area of the binder: 377 m²/g)

Pore volume: 0.43 cm3/g (by means of mercury porosimetry)

Methane absorption: 52 g/l (at 298 K, 50 bar)

### Example 2

### Spheronizing with 20% by Weight of Bentonite

Aluminum fumarate MOF (1000 g) was initially charged in an Eirich intensive mixer (model: R02, RV02). Bentonite (250 g) was added and mixed with the MOF. A manual pressure sprayer was used to spray on demineralized water (2069 g) with continuous movement of the mixture over 30 minutes. Within this time, a second portion of aluminum fumarate MOF (70 g) was added. After completing the addition of water, the spherical shaped bodies formed were dried (12 h, 100° C.) and activated (5 h, 200° C.). 1038 g of spheres were obtained.

Diameter: 4-15 mm

Bulk density of sphere packing: 0.48 g/ml

Average density of spheres: 0.47-1.09 g/ml

Crush strength: 64 N

Langmuir surface area: 966 m2/g (surface area of the binder: 508 m²/g)

Pore volume: 0.54 cm3/g (by means of mercury porosimetry)

Methane absorption: 50 g/l (at 298 K, 50 bar)

### Example 3

### Spheronizing with 20% by Weight of Pural SB

Aluminum fumarate MOF (1000 g) was initially charged in an Eirich intensive mixer (model: R02, RV02). Pural SB (250 g) was added and mixed with the MOF. A manual pressure sprayer was used to spray on a mixture of formic acid (7.5 g) and demineralized water (100 g) with continuous movement of the mixture. Thereafter, pure demineralized water (1795 g) was sprayed on with continuous movement of the mixture over 35 minutes. After completing the addition of water, the spherical shaped bodies formed were dried (12 h, 100° C.) and activated (5 h, 200° C.). 900 g of spheres were obtained.

Diameter: 2-8 mm

Bulk density of sphere packing: 0.39 g/ml

Average density of spheres: 0.27-1.15 g/ml

Crush strength: 24 N

Langmuir surface area: 812 m2/g (surface area of the binder: 381 m²/g)

Pore volume: 0.54 cm3/g (by means of mercury porosimetry)

Methane absorption: 50 g/l (at 298 K, 50 bar)

### Example 4

### Spheronizing with 3% by weight of Secar 80 Cement

Aluminum fumarate MOF (1000 g) was initially charged in an Eirich intensive mixer (model: R02, RV02). Secar 80 cement (30 g) was added and mixed with the MOF. A manual pressure sprayer was used to spray on demineralized water (1895 g) with continuous movement of the mixture over 50 minutes. After completing the addition of water, the spherical shaped bodies formed were dried (12 h, 100° C.) and activated (5 h, 200° C.). 910 g of spheres were obtained.

Diameter: 4-9 mm

Bulk density of sphere packing: 0.38 g/ml

Average density of spheres: 0.49-0.87 g/ml

Crush strength: 35 N

Langmuir surface area: 1078 m2/g (surface area of the binder: 107 m²/g)

Pore volume: 0.66 cm3/g (by means of mercury porosimetry)

Methane absorption: 51 g/l (at 298 K, 50 bar)

### Example 5

### Spheronizing without Additive

Aluminum fumarate MOF (1000 g) was initially charged in an Eirich intensive mixer (model: R02, RV02). A manual pressure sprayer was used to spray on demineralized water (1900 g) with continuous movement of the mixture over 50 minutes. After completing the addition of water, the spherical shaped bodies formed were dried (12 h, 100° C.) and activated (5 h, 200° C.). 735 g of spheres were obtained.

Diameter: 3-8 mm

Bulk density of sphere packing: 0.39 g/ml

Average density of spheres: 0.46-1.21 g/ml

Crushing strength: 34 N

Langmuir surface area: 1138 m2/g

Pore volume: 0.68 cm3/g (by means of mercury porosimetry)

Methane absorption: 52 g/l (at 298 K, 50 bar)

1. A method for preparing a shaped body in the form of spheres comprising:
mixing a composition comprising the MOF and at least one liquid.

2. The method of claim 1 comprising mixing a composition comprising the MOF, the at least one liquid and at least one additive.

3. The method of claim 2, wherein the at least one additive comprises a binder selected from the group consisting of inorganic oxides, aluminum oxide, clays, bentonite and concrete.

4. The method of claim 3, wherein the amount of the at least one binder additive based on the total weight of the shaped body is from 1 to 80 wt.-%.

5. The method of claim 4, which further comprises heating the mixture at a temperature of 100° C. or less.

6. The method of claim 2, wherein the at least one additive comprises a pore forming agent selected from the group consisting of organic polymers.

7. The method of claim 6, wherein the organic polymer is selected from the group consisting of methylcellulose and polyethylene oxide, or mixtures thereof.

8. The method of claim 6, which further comprises, an activation step at a temperature of 300° C. or less.

9. The method of claim 1, wherein a metal of the MOF is selected from the group consisting of Mg, Zn, and Al, or mixtures thereof.

10. The method of claim 9, wherein the MOF comprises
aluminum; and
fumarate, trimesate, 2-aminoterephthalic acid or 4,4′,4″-benzene-1,3,5-triyltribenzoate or mixtures thereof.

11. The method of claim 1, wherein the spheres have diameters in the range of from 1 to 50 mm.

12. A shaped body in the form of spheres produced by the method of claim 3.

13. A shaped body in the form of spheres produced by the method of claim 1.

14. A method for the uptake of at least one substance for the purposes of its storage, separation, controlled release, chemical reaction or as support, comprising:
providing a shaped body of claim 12, and
contacting the shaped body with the at least one substance.

15. The method of claim 14, which further comprises, preparing the shaped body by mixing a composition comprising a MOF and at least one liquid and heating the mixture at a temperature of 100° C. or less. for the uptake of at least one substance for the purposes of its storage, separation, controlled release, chemical reaction or as support.

16. The method of claim 14, wherein the at least one substance is a gas or gas mixture.

17. The method of claims 15, wherein the at least one substance is natural gas or shale gas

18. The method of claim 16, wherein the shaped body is introduced into a vehicle tank, a gas container, or a storage volume of a gas transporter vehicle, and is brought into contact with the at least one substance for storage in the vehicle tank, gas container, or storage volume of the gas transporter vehicle.
